(19) United States
(12) Patent Application Publication
(10) Pub. No.: US 2003/0004464 A1
(43) Pub. Date: Jan. 2, 2003

(54) Chest catheter water barrier

(76) Inventor: Betty Leah Weiner, Farmington Hills, MI (US)

Correspondence Address: JOHN G. CHUPA, Esq., SUITE 205, 31313 NORTHWESTERN HIGWAY, FARMINGTON HILLS, MI, 48334, US

(21) Appl. No.: 09/884,177

(22) Filed: Jun. 18, 2001

(51) Int. Cl. A61M 5/32

(52) U.S. Cl. 604/179

(57) ABSTRACT

A catheter water barrier 10 having a waterproof barrier portion 12 having a pair of channels 16,18 and a relatively long tie down strip 14 which is disposed through the pair of channels 16, 18. The catheter water barrier 10 is worn by a user 17 having a catheter 15 and which provides a watertight seal against the user 17 to protect the region around the catheter from becoming wet.

### FIELD OF THE INVENTION

The present invention generally relates to a chest catheter water barrier and more particularly, to a substantially waterproof chest catheter water barrier which is selectively wearable by a user and which is effective to reduce the likelihood of infection or discomfort to the user of a chest catheter.

### BACKGROUND OF THE INVENTION

A central line catheter or chest catheter generally comprises a substantially plastic tube which is inserted into a large vein of an individual's chest. These chest catheters are used to transfer various intravenous fluids (e.g., chemotherapy solutions) to an individual and to allow blood to be obtained from the individual. In order to facilitate this transfer of intravenous fluid, one open end portion of the chest catheter must project out of the user's chest. Due to the fact that chest catheters involve maintaining an exit site out of the chest, a user must be particularly careful to keep the projecting catheter and the catheter exit site clean and dry to avoid infection.

Because the exit site and catheter need to be kept dry, showering presents difficulties to a person with a chest catheter. Current methods and devices which permit a person with a chest catheter to shower involve applying a sheet of waterproof material to the person's chest over the catheter and using an adhesive to attach the material to the person's skin (i.e., applying tape around edges of the sheet).

While these current methods and devices are intended to prevent water from reaching the catheter and/or catheter exit site, they do have several drawbacks. One drawback is that the tape or adhesive oftentimes loses its adhesive ability when water is applied to it and subsequently, water will enter or intrude upon the area sought to be protected.

Another drawback to applying a waterproof sheet using an adhesive is that individuals who require chest catheters are often in a relatively fragile physical condition. This is particularly so with individuals who are receiving chemotherapy (oftentimes through the chest catheter). The chemicals used in chemotherapy often result in side effects such as, but not limited to, extreme sensitivity and fragility of the skin. By using an adhesive to hold the sheet to the body which must later be pulled off or “torn away” from the wearer's skin/chest, current methods and devices cause great discomfort and/or pain to the individual it is applied to.

Therefore, there is a need for a new and improved substantially waterproof catheter water barrier which overcomes at least some of the previously delineated drawbacks of prior methods and devices, which provides an effective and safe means to keep a chest catheter and its exit site dry, and which may be removed from a user with no resulting physical discomfort or pain.

### SUMMARY OF THE INVENTION

It is a first advantage of the present invention to provide a catheter water barrier which overcomes at least some of the previously delineated drawbacks of prior catheter waterproofing devices.

It is a second advantage of the present invention to provide a catheter water barrier which overcomes at least some of the previously delineated drawbacks of prior catheter waterproofing devices and which, by way of example and without limitation, provides a waterproof barrier which may be tied or strapped on to a wearer.

It is a third advantage of the present invention to provide a catheter water barrier which overcomes at least some of the previously delineated drawbacks of prior catheter waterproofing devices and which substantially prevent water from being introduced to the protected region.

According to a first aspect of the present invention, a catheter water barrier is provided. The catheter water barrier comprises a section of waterproof material having two channels along opposing edges and a relatively long tie down strip which is disposed through the two channels.

According to a second aspect of the present invention, a catheter water barrier is provided. The catheter water barrier comprises a generally rectangular waterproof polymer barrier portion having two channels and a relatively long nylon tie strip which is disposed through the two channels.

According to a third aspect of the present invention a method for using a catheter water barrier on a user having a chest catheter is provided. The method comprises the steps of providing a catheter water barrier having a barrier portion, at least two channels, and a relatively long tie down strip; forming a loop by inserting the tie down strip through the at least two channels; placing the head of the user through the loop; disposing the barrier portion over the chest catheter; and tying the ends of the tie down strip together, thereby causing the tie down strip to hold the barrier portion to the user.

These and other features, aspects, and advantages of the present invention will become apparent from reading the following detailed description of the preferred embodiment of the invention and by reference to the following drawings.

### BRIEF DESCRIPTION OF THE DRAWINGS

FIG. 1 is a perspective view of a catheter water barrier which is made in accordance with the teachings of the preferred embodiment of the invention and shows the catheter water barrier being worn by an individual with a chest catheter.

FIG. 2 is a front view of the catheter water barrier shown in FIG. 1.

FIG. 3 is a rear view of a catheter water barrier which is made in accordance with the teachings of a second embodiment of the invention and which depicts the barrier portion having a pair of flap which may be placed around a user's catheter.

FIG. 4 is a front view of a catheter water barrier which is made in accordance with the teachings of a third embodiment of the invention and which depicts the channels forming an “X”-shaped pattern across the barrier portion.

### DETAILED DESCRIPTION OF THE PREFERRED EMBODIMENT OF THE INVENTION

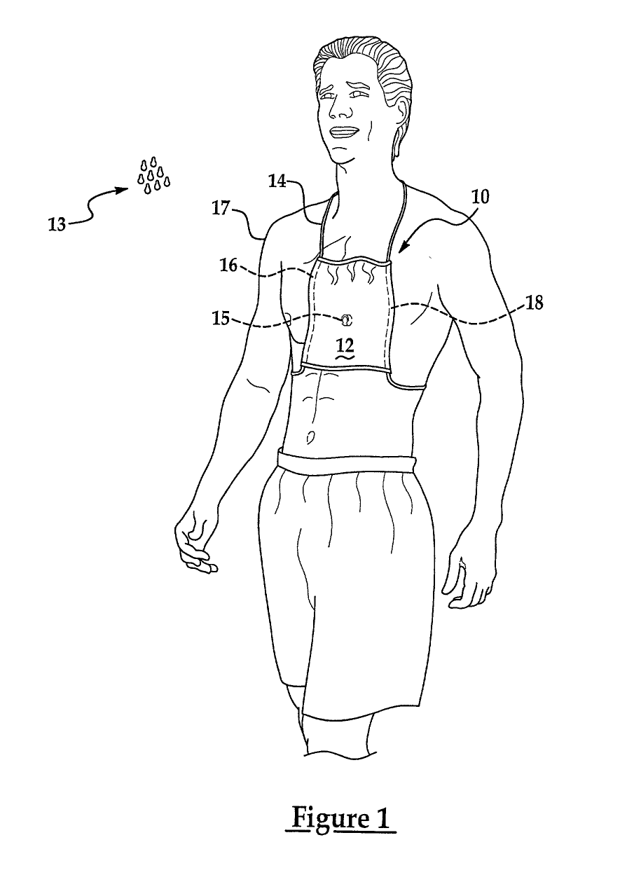

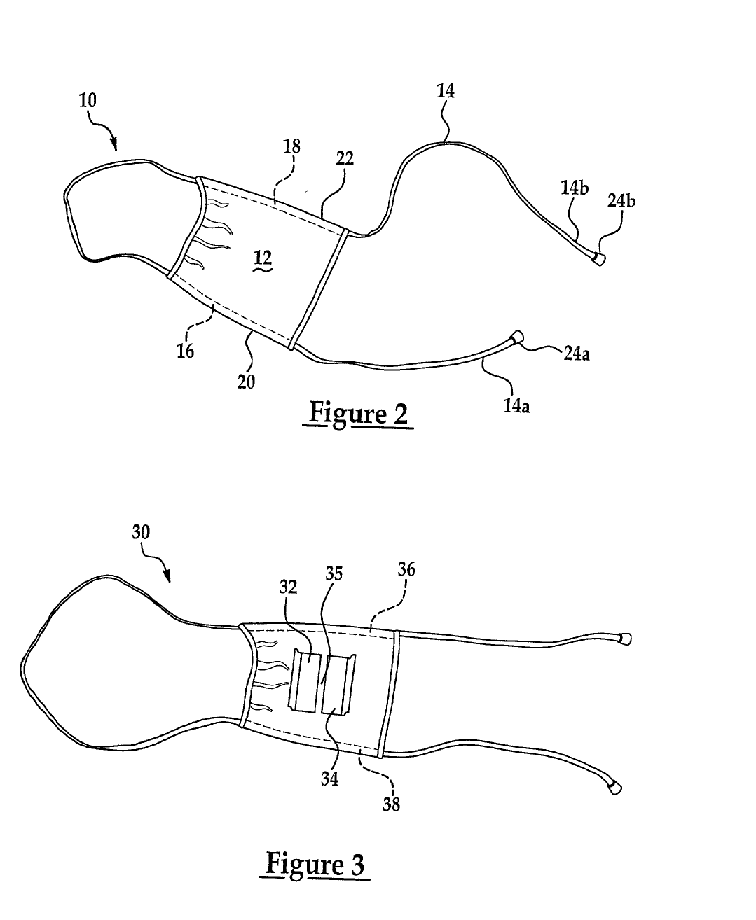

Referring now to FIGS. 1 and 2, there is a catheter water barrier 10 which is made in accordance with the teachings of the preferred embodiment of the invention. Particularly, the catheter water barrier 10 includes a barrier portion 12 and a tie down strip 14. Barrier portion 12 and strip 14 cooperatively act to form a watertight barrier against the intrusion of water 13 or any other outside possible contaminant (e.g., soap or shampoo) into the region around the chest catheter 15 of an individual user 17.

Barrier portion 12 is comprised of a generally rectangular section of relatively thin, substantially waterproof, and non-toxic material such as, but not limited to, a polymer material. In the preferred embodiment of the invention, barrier portion 12 further comprises a pair of channels 16, 18 which are formed along opposing edges 20, 22. Channels 16, 18 may be created by folding over and sealing edges 20, 22 (e.g., by using a conventional adhesive) or alternatively, may be integrally formed within the barrier portion 12.

Tie down strip 14 is a relatively long thin piece of material which, in one non-limiting embodiment, is constructed of a nylon material. Tie down strip 14 is of sufficient length to allow strip 14 to be placed through channel 16, around the neck of a user 17, and through channel 18, while allowing barrier portion 12 to remain over the catheter 15, and while leaving sufficient length to allow the opposing ends 14a, 14b to be tied together behind a user's back (as shown in FIG. 1) or to be wrapped around the back of the user 17 and tied in front of the 5 user 17 (not shown). Tie down strip 14 further comprises a pair of caps 24a, 24b which are disposed upon the respective ends 14a, 14b to prevent the tie down strip 14 from being separated from or “pulled out” of channels 16, 18 of barrier portion 12. In one non-limiting embodiment, caps 24a, 24b are physically coupled to ends 14a, 14b are integrally formed with the down strip 14.

When catheter water barrier 10 is fastened securely to a user 17. The tie down strip 14 acts to physically hold barrier portion 12 against the skin of user 17 and thereby creates a watertight seal to prevent water 13 from reaching catheter 15.

In one non-limiting embodiment of the invention, shown in FIG. 3, catheter water barrier 30 is comprised of substantially all the elements of catheter water barrier 10, but further includes a set of flaps 32, 34 which forms a pouch portion 35 which may be used to substantially envelop catheter 15 to provide additional protection against the intrusion of water 13. Flaps 32, 34 may be attached to barrier portion 12 using a conventional adhesive or alternatively, may be integrally formed with barrier portion 12. The longitudinal axes of flaps 32, 34 are positioned substantially perpendicular to channels 36, 38. It should be noted, however, that while the embodiment shown in FIG. 3 depicts the longitudinal axes of flaps 32, 34 substantially perpendicular to channels 36, 38, alternate embodiments (not shown) may include having flaps 32, 34 running substantially parallel to channels 36, 38.

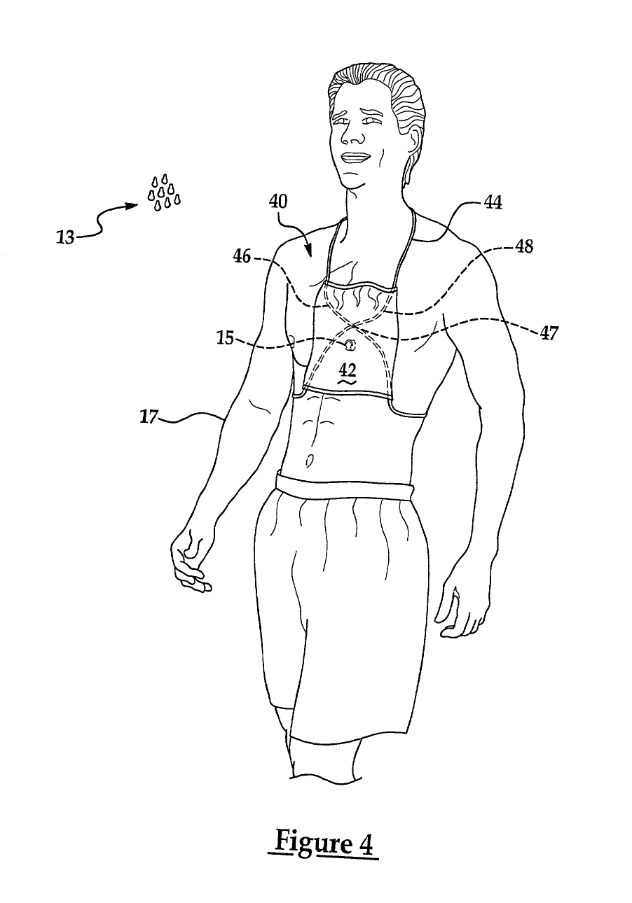

In another non-limiting embodiment of the invention, as shown in FIG. 4, catheter water barrier 40 is substantially similar to catheter water barrier 10, but instead of having channels 16, 18 run along opposing edges 20, 22, catheter water barrier comprises a pair of channels 46, 48 which form an “X”-shaped pattern across the barrier portion 42. In this alternate embodiment, channels 46, 48 intersect at the approximate center of generally rectangular barrier portion 42. As shown in FIG. 4, by placing the intersection point 47 of channels 46, 48 above the position of catheter 15, this embodiment provides additional protection against intrusion by water 13 when tie down strip 44 is tied tightly and thereby causes strip 44 to push barrier portion 42 securely against the skin of user 17.

It is to be understood that the invention is not limited to the exact construction or method which has been previously delineated, but that various changes and modifications may be made without departing from the spirit and the scope of the inventions as are set forth in the following claims. Examples of such modifications include, but are not limited to: providing additional tie down strips 14; applying a barrier cream (e.g., petroleum jelly) substantially around the catheter 15 to cooperate with the barrier portion 12; or increasing the rigidity of the edges orthogonal to edges 20, 22. It should be appreciated that barrier 10 may be selectively used to cover substantially any part of the body and that nothing in this Application is meant to limit the use of the barrier 10 to only the chest area.

What is claimed is:

1) A catheter water barrier comprising a section of waterproof material having two channels along opposing edges and a relatively long tie down strip which is disposed through said two channels.

2) The catheter water barrier of claim 1 wherein said section of waterproof material is generally rectangular in shape.

3) The catheter water barrier of claim 1 wherein said catheter water barrier further comprises a pair of flaps having longitudinal axes, said flaps being coupled to said section of waterproof material to provide a pouch portion.

4) The catheter water barrier of claim 3 wherein said longitudinal axes of said pair of flaps are substantially perpendicular to said channels.

5) The catheter water barrier of claim 3 wherein said longitudinal axes of said pair of flaps are substantially parallel to said channels.

6) The catheter water barrier of claim 1 wherein said section of waterproof material is formed from a polymer material.

7) The catheter water barrier of claim 1 wherein said tie down strip is formed from a nylon material.

8) A catheter water barrier comprising a generally rectangular waterproof polymer barrier portion having two channels and a relatively long nylon tie strip which is disposed through said two channels.

9) The catheter water barrier of claim 8 wherein said two channels are disposed along opposing edges of said barrier portion.

10) The catheter water barrier of claim 8 wherein said two channels form an “X”-shaped pattern across said barrier portion.

11) The catheter water barrier of claim 10 wherein said two channels intersect at the approximate center of said barrier portion.

12) A method for using a catheter water barrier on a user having a chest catheter is provided, said method comprises the steps of:
providing a catheter water barrier having a barrier portion, at least two channels, and a relatively long tie down strip;
forming a loop by inserting said tie down strip through said at least two channels;
placing the head of said user through said loop;
disposing said barrier portion over said chest catheter; and
tying the ends of said tie down strip together, thereby causing said tie down strip to hold said barrier portion to said user.

13) The method of claim 12 further comprising the steps of:
forming a pair of flaps upon said barrier portion to form a pouch portion; and
placing said chest catheter within said pouch portion.

14) The method of claim 12 wherein said step of tying the ends of said tie down strip together, thereby causing said tie down strip to hold said barrier portion to said user further comprises the step of wrapping said ends of said tie down strip around the back of said user.

15)The method of claim 12 wherein said at least two channels of said barrier portion are formed in a “X”-shaped pattern across said barrier portion and wherein said step of disposing said barrier portion over said chest catheter further comprises the step of placing the intersection of said “X”-shaped pattern above said chest catheter.
